(19) United States
(12) Patent Application Publication
(10) Pub. No.: US 2024/0315544 A1
(43) Pub. Date: Sep. 26, 2024

(54) Otoscope Attachment for Enhanced Visualization with Mobile Devices

(71) Applicants: Haluk Un, WOODINVILLE, WA (US); MUSTAFA YASAR SIR, WOODINVILLE, WA (US)

(72) Inventors: Haluk Un, WOODINVILLE, WA (US); MUSTAFA YASAR SIR, WOODINVILLE, WA (US)

(21) Appl. No.: 18/609,066

(22) Filed: Mar. 19, 2024

Related U.S. Application Data
(60) Provisional application No. 63/453,339, filed on Mar. 20, 2023.

(51) Int. Cl. A61B 1/227; A61B 1/00; A61B 1/04; A61B 1/06; A61B 1/32

(52) U.S. Cl. CPC A61B 1/227; A61B 1/000095; A61B 1/000096; A61B 1/00105; A61B 1/0011; A61B 1/0014; A61B 1/042; A61B 1/0669; A61B 1/32

(57) ABSTRACT

The invention discloses an otoscope device attachment intended for use with image capture devices featuring illumination capabilities. This device attachment comprises a connecting component and a speculum. The connecting component is designed to detachably secure to a user device, featuring a planar body with a first side equipped with means for attachment to the user device and an opposing second side. A central aperture is formed within the planar body of the connecting component to expose the camera of the user device. The speculum, characterized by an elongated body with an outer and an inner surface, defines a straight tunnel extending from its proximal end, which forms a base to detachably couple to the second side of the connecting component, to its distal end, forming a tip. Positioned along the length of the straight tunnel within the speculum are one or more lenses.

### FIELD OF INVENTION

The present invention relates generally to the field of medical diagnostic devices, specifically to an attachment for otoscopes designed to facilitate the examination of the ear and other body orifices using image capture and illumination capabilities of mobile devices.

### BACKGROUND

The contemporary medical landscape is increasingly reliant on advanced diagnostic tools for efficient and accurate examination of patient conditions, particularly in the realm of telemedicine and remote diagnostics. A critical component of these diagnostic tools is the otoscope, traditionally used in clinical settings for the examination of the ear canal and tympanic membrane. Despite the advancements in medical technology, there exist significant limitations and drawbacks with the current generation of otoscopes and similar systems, particularly when considering their integration with modern image capture and communication devices such as smartphones.

One of the primary challenges faced by medical professionals and patients alike is the difficulty in obtaining clear, well-illuminated images of the inner and outer structures of organs, such as the ear, during examinations. This challenge is further exacerbated in telemedicine settings, where physical proximity to the patient is not possible. Traditional otoscopes, while effective in a clinical setting, are often unsuitable for use by patients or in remote settings due to their complex design, the requirement for direct handling by medical professionals, and the need for specialized, often costly, equipment.

Furthermore, the existing devices designed to overcome these challenges typically operate as standalone electronic devices, necessitating the use of multiple devices simultaneously for communication and examination. This not only increases the logistical complexity of conducting remote examinations but also places a financial burden on patients and healthcare providers due to the need for additional, often expensive, equipment. Additionally, the compatibility of these devices with a wide range of smartphones and tablets remains a significant hurdle, as many are designed to work with specific models or brands, limiting their applicability and accessibility.

Moreover, the integration of multiple cameras in modern electronic devices introduces uncertainty regarding the optimal setup for using otoscope attachments, affecting the consistency and quality of the images captured. The quality of the data captured during examinations is highly dependent on the stability of the device, the positioning of the camera relative to the subject, and the ability of the device to adequately illuminate the area being examined. These factors underscore the need for a versatile, user-friendly attachment that can be universally adapted to different electronic devices, ensuring high-quality image capture while mitigating the limitations associated with traditional otoscopes and existing electronic attachment designs.

Addressing these challenges is crucial for enhancing the efficiency and effectiveness of remote medical examinations, reducing the dependency on in-person clinical visits for ear examinations, and expanding the accessibility of high-quality healthcare services. The development of a new attachment device for otoscopes represents a significant step forward in achieving these objectives, offering a solution that is not only accessible and cost-effective but also compatible with a broad range of modern electronic devices.

Several solutions in the realm of otoscope devices and their integration with mobile electronic devices have been identified, each presenting certain limitations that the current invention seeks to overcome. For instance, patents such as U.S. Pat. No. 9,325,884B2, US20130102359A1, US20200029837A1, and KR101349923B1 describe otoscope apparatuses with illumination direction elements in fixed positions, rendering them incompatible with mobile devices that have light sources located in various positions. This fixed positioning limits the versatility and adaptability of these devices when used with a broad range of smartphones and tablets, which often feature different designs and configurations of cameras and light sources.

Furthermore, patents like U.S. Pat. No. 11,246,489B2, US20210068645A1, US20120245422A1, and WO2013156999A1 detail otoscope designs that are characterized by their complexity and the requirement for multiple optical and electronic components. These aspects contribute to higher production costs and a user experience that is less than intuitive, potentially discouraging widespread adoption and use, particularly in settings where ease of use and cost-effectiveness are paramount.

The complex design mentioned in patents such as US20210068645A1, US20120245422A1, and WO2013156999A1 also signifies a barrier to accessibility. The intricacy of these devices not only affects their ease of use but also escalates the manufacturing expenses, thus affecting the final cost to the end user. This complexity could limit the practicality of deploying such devices in resource-limited settings or among non-professional users who may benefit from conducting preliminary examinations without the need for specialized training.

It is within this context that the present invention is provided.

### SUMMARY

The present invention relates to an otoscope device attachment designed for use with image capture devices that have illumination capabilities. This device attachment includes a connecting component for detachably securing to a user device and a speculum designed to be inserted into an orifice for medical examination purposes. The connecting component houses a central aperture for camera exposure, while the speculum, equipped with one or more lenses, aligns with the device's camera to facilitate illuminated visualization of internal structures through a combination of transparency and internal light reflection.

In some embodiments, the connecting component can attach to the user device using various means such as adhesive materials, suction cups, or clip-shaped structures. This flexibility in attachment means allows the device to be universally applicable to a wide range of user devices, enhancing its practicality and ease of use in diverse settings.

The speculum in certain embodiments is composed of an inner and an outer body, creating a hollow space between them that aids in the internal reflection of light. This design enhances the illumination within the examined orifice, improving the quality of the images captured.

In further embodiments, the material composition of the speculum's inner and outer bodies can vary, including plastic, metal, silicone, and glass, and can be transparent, translucent, or opaque. This variation allows for the optimization of light transmission and reflection, catering to different examination requirements.

Additionally, the outer wall of the inner body and the inner wall of the outer body possess reflective properties in some embodiments. This feature significantly enhances the efficiency of light transmission along the length of the speculum, ensuring that the examination area is adequately illuminated.

The lenses within the speculum can be convex, concave, or a combination thereof, according to some embodiments. These lenses are configured to transmit images from the tip of the speculum to the user device's camera clearly. Moreover, these lenses may be coated with various light filters to further enhance the quality of transmitted images.

In some embodiments, the speculum's base is wider than its tip, adopting a tapered shape that facilitates easy insertion into various body orifices. This design feature enhances the user's comfort and the overall usability of the device.

The connecting component is also designed to be compatible with electronic devices of varying sizes and shapes, ensuring proper alignment of the camera with the central aperture regardless of the device model. This universality extends the device's applicability across a wide range of smartphones and tablets.

Furthermore, the connecting component includes shapes and apertures specifically designed to match the camera's image area. This feature ensures that the camera effectively captures images through these apertures while allowing the passage of light from the device's light source.

A mechanism within the connecting component allows for the selection of the appropriate camera on devices equipped with multiple cameras. This adaptability ensures the otoscope device attachment's compatibility with modern multi-camera devices, optimizing its functionality.

In certain embodiments, the speculum incorporates a soft material at its distal end, designed to safely and comfortably enter an orifice without causing harm. This consideration for patient comfort and safety is critical in medical examinations.

The connecting component is equipped with multiple connection points for attaching a variety of medical examination accessories, according to some embodiments. This versatility transforms the device into a multifunctional diagnostic tool capable of conducting a broader range of examinations.

A rotatable platform on the connecting component, present in some embodiments, enables the precise positioning of different accessories in alignment with the camera opening and the light source. This feature facilitates seamless switching between examination functions without the need for detaching and reattaching accessories.

In another embodiment, the connection part includes a mechanism for linear movement, allowing for the adjustment of the camera and light source opening or closing. This adjustability enhances the device's versatility in various examination scenarios.

Integrated with artificial intelligence models, the otoscope device attachment in certain embodiments can automatically adjust stability, lighting, distance, and zoom to optimize image capture. The inclusion of AI models for image quality assessment, patient positioning guidance, and image resolution and clarity enhancement marks a significant advancement in medical diagnostic technology, offering a sophisticated, user-friendly solution for a wide array of examination needs.

### BRIEF DESCRIPTION OF THE DRAWINGS

Various embodiments of the invention are disclosed in the following detailed description and accompanying drawings.

FIG. 1A is a perspective view of an example configuration of the speculum.

FIG. 1B is a perspective view of an example configuration of the speculum.

FIG. 2A is a side-sectional view of an example configuration of the speculum.

FIG. 2B is a bottom view of an example configuration of the speculum.

FIG. 3A is a side-sectional view of an example configuration of the speculum.

FIG. 3B is a bottom view of an example configuration of the speculum.

FIG. 4A is a top view of an example configuration of the connecting part.

FIG. 4B is a side view of an example configuration of the connecting part.

FIG. 5A is a perspective view of an example configuration of the electronic device.

FIG. 5B is a perspective view of an example configuration of the connecting part mounted on the electronic device.

FIG. 6 is a perspective view of an example configuration of the speculum, and connecting part mounted on the electronic device.

FIG. 7 is a side-sectional view of an example configuration of the speculum, and connecting part mounted on the electronic device.

FIG. 8A is a perspective view of an example configuration of the connecting part.

FIG. 8B is a perspective-sectional view of an example configuration of the connecting part.

FIG. 9A is a side-sectional view of an example configuration of the speculum.

FIG. 9B is a side-sectional view of an example configuration of the connection part mounted on the electronic device.

FIGS. 9C and 10A are side-sectional views of an example configuration of the speculum, and connecting part mounted on the electronic device.

FIG. 10B is a perspective view of an example configuration of the connecting part.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

The use of spatial terms, such as “under,” “below,” “lower,” “over,” “upper,” etc., is used for ease of explanation to describe the relationship between elements when the apparatus is in its proper orientation.

The terms “first,” “second,” and the like are used to distinguish different elements or features, but these elements or features should not be limited by these terms. A first element or feature described can be referred to as a second element or feature and vice versa without departing from the teachings of the present disclosure.

As used herein, “image capture devices comprising illumination capabilities” refers to any electronic device equipped with both a camera for capturing images or video and an integrated light source, such as an LED flash or other lighting technology, designed to illuminate the subject being captured. Example devices include smartphones, tablets, and digital cameras that are commonly used for communication and multimedia tasks. These devices must possess software and hardware interfaces that enable the attachment and integration of external accessories like the otoscope device attachment described herein.

The term “connecting component” as used herein, encompasses any structure or assembly that facilitates the physical and functional connection between the otoscope device attachment and the user device. This component can be realized in various forms, including but not limited to, snap-fit mechanisms, magnetic attachments, adhesive pads, or clamping devices. The design of the connecting component allows for easy attachment and removal from the user device without causing damage or permanent alteration to the device.

“Speculum” as described herein, refers to a medical instrument traditionally used to investigate body orifices, modified in this context to attach to and work in conjunction with the described otoscope device attachment. The speculum includes a tunnel-like structure through which the camera's view is directed, and may incorporate features such as adjustable diameters or lengths to accommodate different examination requirements and patient comfort levels.

As used herein, “lenses” can include single or multiple optical elements made from glass, plastic, or any transparent material capable of focusing or altering the path of light. These lenses are specifically arranged to optimize the clarity and focus of the images captured by the camera through the speculum. The lenses can have different focal lengths, shapes (convex, concave), and coatings (anti-reflective, filter) to enhance image quality under various lighting conditions and examination scenarios.

### DESCRIPTION OF DRAWINGS

The present invention relates to a device attachment designed to transform conventional image capture devices with illumination capabilities, such as smartphones and tablets, into functional otoscopes. This innovative attachment offers a practical solution for conducting ear examinations and visualizing inner and outer structures of body orifices without the need for specialized medical equipment. The invention consists primarily of two components: a connecting component and a speculum.

The connecting component serves as the intermediary between the user device and the speculum, ensuring a secure and detachable connection. It features a planar body designed with a first side for attachment to the user device, utilizing various means such as adhesives, suction cups, or clips for versatile compatibility with different device models and sizes. The opposing second side of the connecting component is configured to couple with the speculum. A central aperture within the planar body is strategically positioned to align with the camera of the user device, facilitating the use of the device's built-in illumination to light the examination area.

The speculum, an integral part of the attachment, is an elongated body that guides the light from the device's illumination source to the area under examination. It features an inner surface defining a straight tunnel that extends from its proximal end, where it connects to the connecting component, to its distal end, which is inserted into the orifice being examined. The speculum is designed to align with the camera through the central aperture of the connecting component, allowing for clear image capture. Embedded within the speculum are one or more lenses, positioned along the tunnel to optimize the quality of the transmitted images. These lenses can be of various types, including convex and concave, and may include coatings or filters to enhance image clarity under different lighting conditions.

The invention leverages the widespread availability and advanced capabilities of modern image capture devices, offering a cost-effective and accessible tool for medical professionals and potentially for individuals conducting self-examinations. By integrating the device's camera and light source, the attachment provides a means to illuminate and visualize areas that are typically difficult to examine, such as the ear canal, thereby facilitating the diagnosis and monitoring of medical conditions.

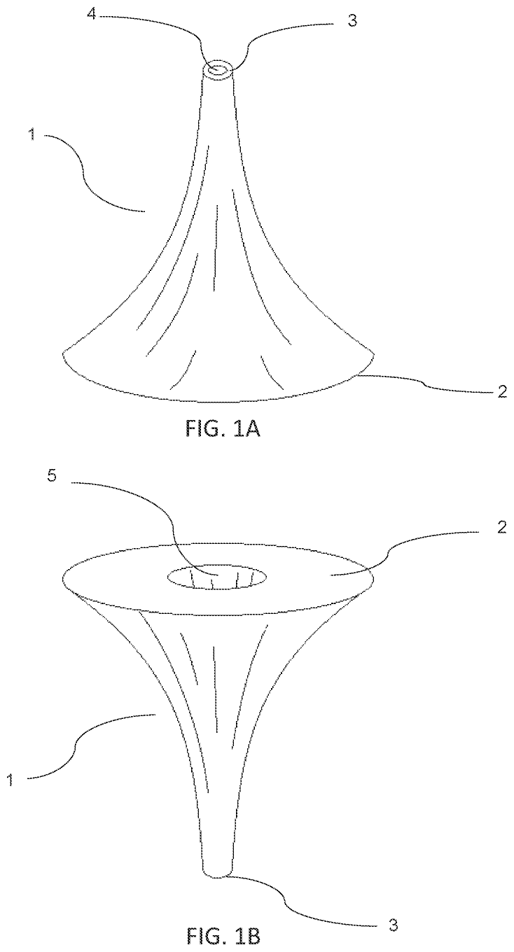

FIG. 1A provides a perspective view of the speculum (1), showcasing its dual-end configuration. The base part (2), intended for attachment to the electronic device (12), is characterized by its broad and flat design. In contrast, the tip (3) is crafted to be narrower and more compact, allowing for seamless insertion into various body orifices, such as the ear or nose. This end may adopt a cone shape, tapering towards the tip opening (4), or resemble a straight tube, and may present either a symmetrical or asymmetrical structure depending on the specific application.

FIG. 1B further illustrates the speculum (1), emphasizing the flat design of its base part (2) which facilitates placement against the electronic device (12). Central to this part is the base opening (5), encircling the camera (13) of the electronic device (12) to enable image capture. This opening can be fashioned in various shapes including circular, elliptical, or an asymmetrical form to accommodate the diversity of electronic device designs.

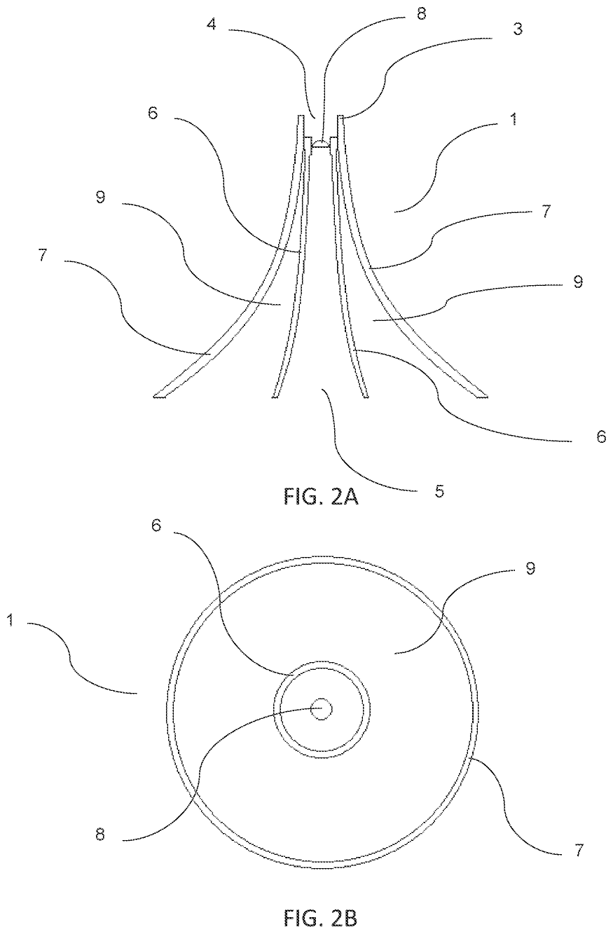

In FIG. 2A, a side view reveals an alternate configuration of the speculum (1), delineating the distinct inner (6) and outer (7) bodies. These components are interconnected, housing one or more lenses (8) within the inner body (6) to focus and transmit light effectively. The lenses (8) may vary in proximity to the device's camera and differ in size at each terminal of the speculum (1), catering to a range of examination depths and focal requirements. The speculum can feature either a continuous structure or a segmented design with a gap (9) between the inner (6) and outer (7) bodies, potentially offering modularity in assembly. The lengths of the inner (6) and outer (7) bodies may also vary, providing flexibility in adapting to different examination scenarios.

FIG. 2B presents a bottom view of the speculum (1), illustrating the alignment of the base opening (5) and the tip opening (4). This arrangement ensures that light rays traverse the speculum and focus through the lenses (8) onto the camera (13), enhancing the clarity and precision of captured images. The speculum (1) may exhibit either a symmetrical circular or an asymmetrical shape, with the option for parts to be transparent, semi-transparent, or opaque. Additionally, a reflective coating may be applied to the speculum's components to optimize light distribution within the examination field.

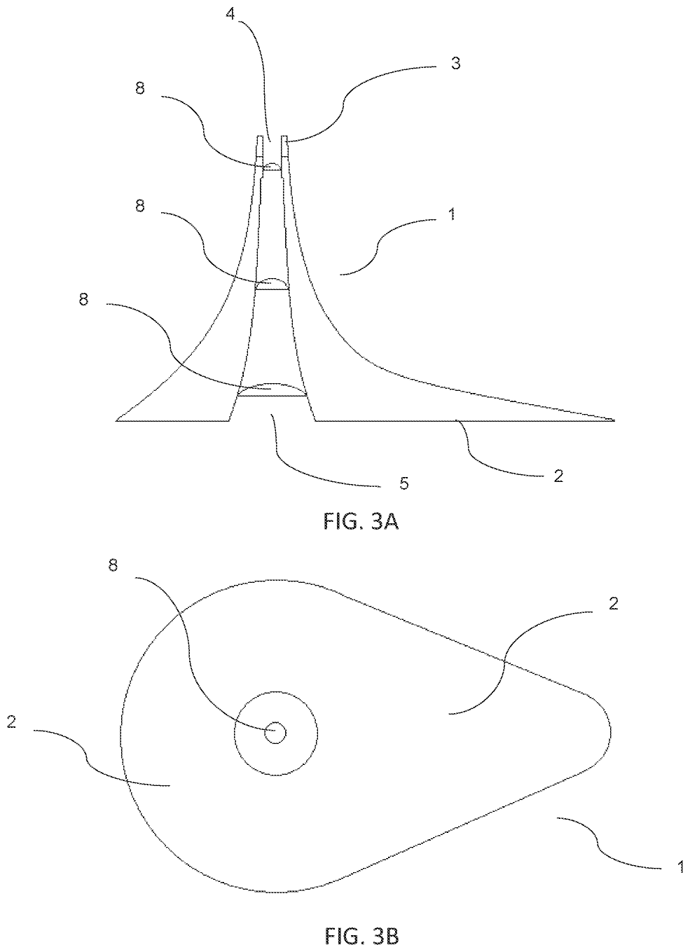

FIG. 3A illustrates a side cross-sectional view of the speculum (1), demonstrating that the speculum can be a unified structure without a gap (9) separating its inner (6) and outer (7) bodies. This cohesive design enhances structural integrity and streamlines light transmission. The distal end, or tip (3), is fabricated from a soft, flexible material and designed with a gentle curve to mitigate any discomfort or potential harm to sensitive body orifices during insertion. The speculum's body may exhibit an asymmetrical configuration, wherein one side extends longer than the opposite, enabling strategic direction of light towards specific areas within the visual field. The material at the tip (3) is specifically chosen for its smoothness, further ensuring patient comfort during use.

In FIG. 3B, the bottom view of the speculum (1) from the perspective shown in FIG. 3A is displayed. Here, the outer body (7) of the speculum (1) adopts an asymmetrical shape, a design choice that allows for versatile alignment with various configurations of cameras (13) and light sources (14) found on different electronic devices. The outer body (7) can be designed in several forms, including circular, elliptical, or polygonal, with distinct edges to accommodate the positioning requirements of the attached device.

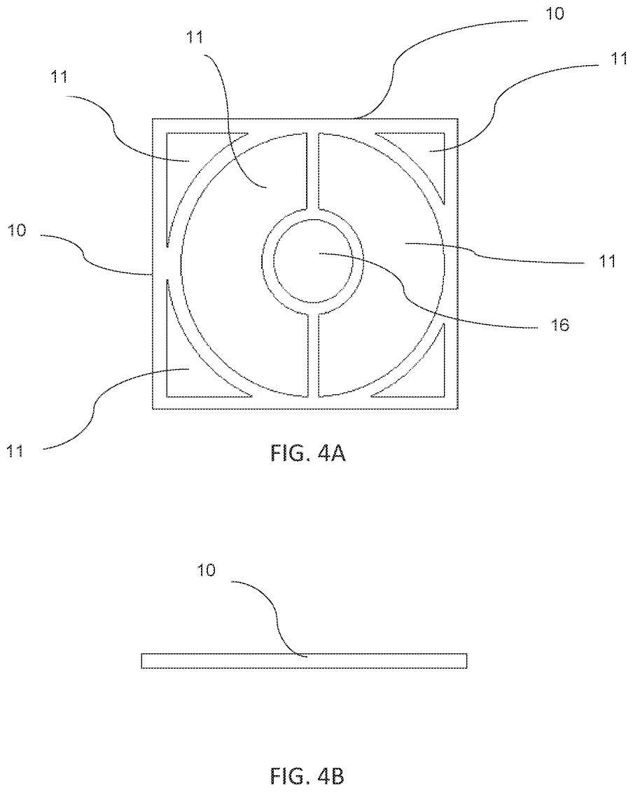

FIG. 4A presents a top view of the connecting part (10), highlighting the presence of strategically placed openings (11). These openings, devoid of material, are essential for the unobstructed passage of light. Their shapes can vary, catering to the needs of specific examinations by directing light precisely onto the area under observation through the speculum (1). Additionally, the central circular opening (16) is specifically designed to encompass the camera area of the electronic device, ensuring that the camera's field of view is optimally aligned with the tunnel of the speculum. The connecting part (10) is characterized by its ability to either reflect or absorb partial light, a feature that prevents interference from the device's light source (14) with the camera's (13) image capture quality. The connecting part's design, including its variable thickness and the material composition of its surfaces, incorporates adhesive properties for secure yet temporary attachment to both the electronic device (12) and the speculum (1).

FIG. 4B offers a side view of the connecting part (10), detailing its variable thickness across different sections to suit various attachment needs and device profiles. The adhesive coating on its surfaces facilitates a firm yet removable bond with the electronic device (12), allowing for easy assembly and disassembly while ensuring stability during use. This adaptability ensures that the otoscope device attachment can be universally applied across a wide range of electronic devices, enhancing its utility in diverse medical settings.

In another embodiment, the connecting part (10) is designed to function as a case that envelops the electronic device (12), offering a versatile solution for attachment. This case structure is engineered with foldable or elastic materials, enabling it to adapt and securely fit electronic devices (12) of varying dimensions and form factors. Alternatively, for a more temporary and adjustable solution, the connecting part (10) can employ vacuum adhesives, adopting a suction cup design that affixes firmly to the electronic device (12) without leaving residue or causing damage. This approach allows for a swift and hassle-free attachment or removal process. Furthermore, the speculum (1) is designed to attach to specific, clearly identified clip-marked areas located on the connecting part (10), ensuring a stable and precise alignment with the device's camera and light source. These clip-marked areas, although not depicted in the illustrations, are integral to facilitating the secure positioning and operational efficacy of the speculum (1) in relation to the connected electronic device (12).

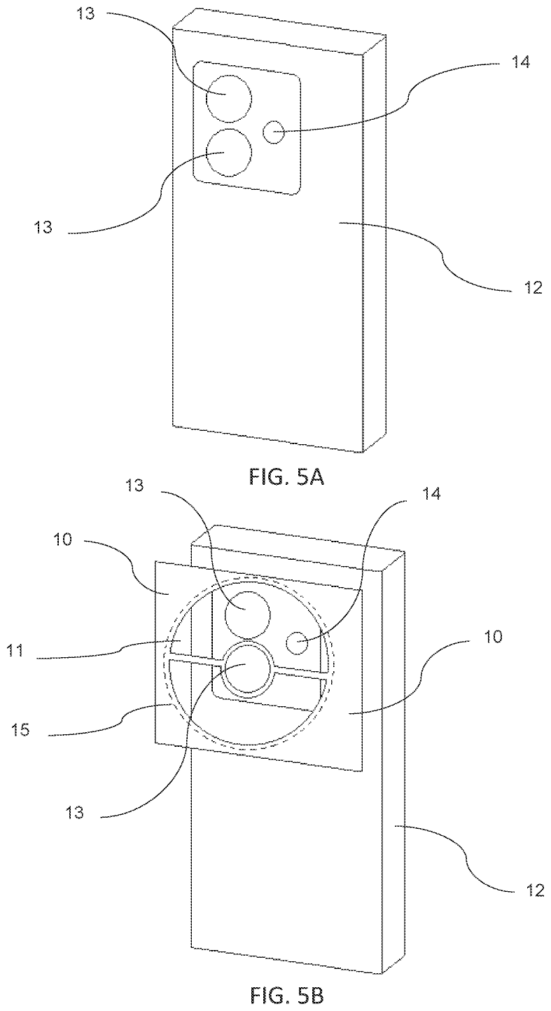

FIG. 5A provides a perspective view of the electronic device (12), illustrating its potential to house multiple cameras (13) positioned across various locations on the device's body. Accompanied by a light source (14), these cameras can have varying proximities to the light source, influencing the illumination and capture of images. The connecting part (10) plays a crucial role in ensuring that the selected camera (13) for image capture is effectively integrated with the device, safeguarding against any adverse effects of excessive lighting from the light source (14) on image quality.

In FIG. 5B, the assembly of the connecting part (10) with the electronic device (12) is showcased. The central circular opening (16) in the connecting part (10) is designed to encircle the camera (13) on the electronic device (12), facilitating precise alignment with the speculum (1) for optimal image capture. This design ensures that light from the light source (14) is unobstructed by the openings (11) on the connecting part (10), permitting it to illuminate the examination area through the speculum (1) effectively. The connection part (10) also features designated regions for the secure attachment of both the inner (6) and outer (7) bodies of the speculum (1). These regions are equipped with either adhesive materials or clip-like mechanisms, allowing for a robust yet reversible connection to the electronic device (12). The structure of the connecting part (10) is versatile, capable of adopting various shapes such as polygonal, elliptical, or circular forms, to complement the geometry of the attached electronic device (12). Additionally, specific areas on the connecting part (10) are designed with suction cup-shaped structures or other adhesive solutions to facilitate a temporary yet secure bond with the electronic device (12), enhancing the overall functionality and user experience of the otoscope device attachment.

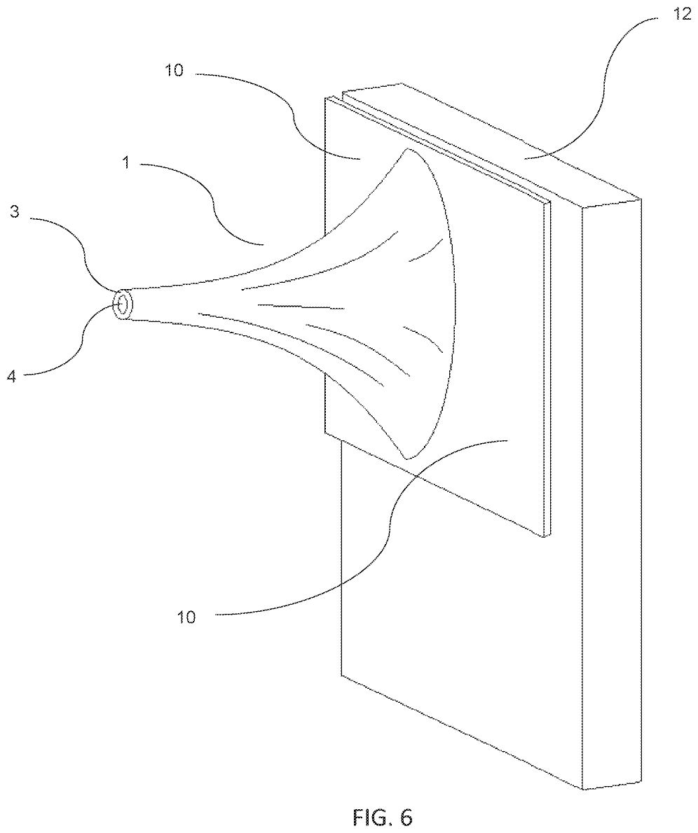

FIG. 6 illustrates the assembly of the speculum (1), connecting part (10), and electronic device (12) as a unified system. The base part (2) of the speculum (1) is precisely positioned on a designated area (15) on the connecting part (10). This arrangement ensures that the wider base part (2) securely interfaces with the connecting part (10), orienting the speculum's tip (3) towards the target examination area. Light, originating from the opening (4) of the speculum (1), is channeled directly towards the electronic device's camera (13) via lenses (8) strategically embedded within the speculum. Concurrently, illumination from the device's light source (14) traverses the connecting part (10), enhancing visibility at the speculum's tip (3) and ensuring the examination area is adequately lit.

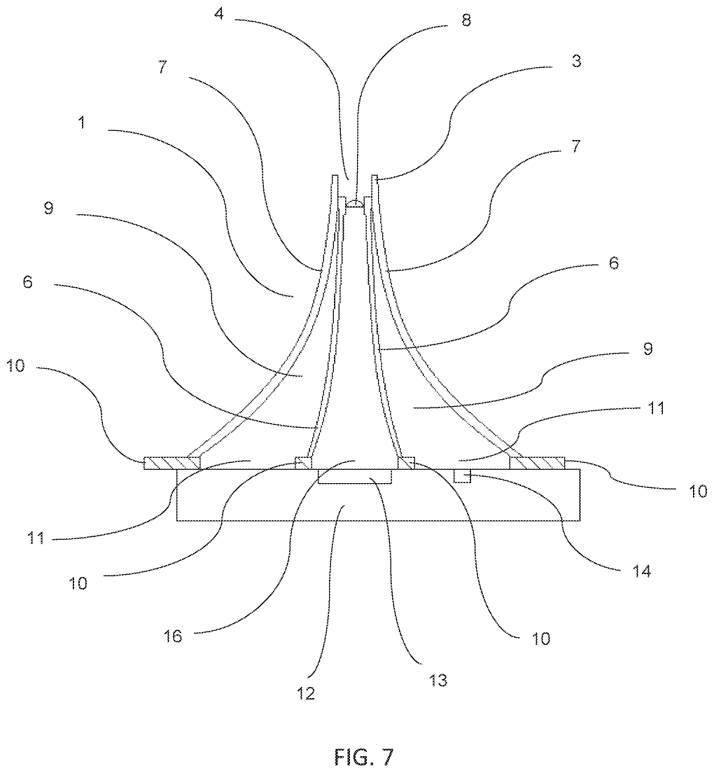

In FIG. 7, a side view of the speculum (1) mounted on the electronic device (12) is depicted. This perspective highlights how the base part (2) of the speculum's inner body (6) envelops the camera (13), optimizing the field of view for image capture. Lenses (8), placed at critical junctures within the speculum, fine-tune the focus and clarity of the images captured by the camera (13). A distinctive gap (9) between the inner (6) and outer (7) bodies facilitates the effective transmission of light from the device's light source (14) to the speculum's tip (3), illuminating the area under inspection. This gap (9) may be encased in materials varying from transparent to partially light-reflective, enhancing light diffusion. The connecting part (10) serves as the intermediary for attaching the speculum to the electronic device (12), featuring openings (11) that permit unimpeded light passage towards the speculum's tip (3). The design of the connecting part (10) allows for versatile attachment methods, either as a stick-on flat connector or as a case-like structure that encases the electronic device (12), providing flexibility in use and application.

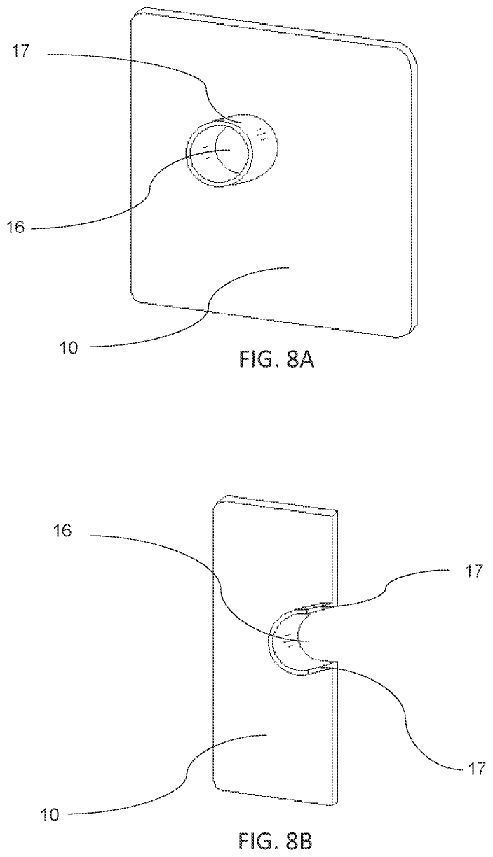

FIG. 8A introduces an alternative configuration of the connecting part (10), designed with a breadth that enables it to encompass potential positions of the light source (14) on various electronic devices (12). This version of the connecting part (10) is characterized by its light-conducting properties, enhancing the illumination capabilities when attached to the device. Featured on the connecting part (10) are extensions (17), protrusions, recesses, and clip-like structures centered around the camera opening (16), which not only serve to secure the speculum (1) but also have the capacity to modulate light-either transmitting it to enhance illumination or reflecting it to minimize glare. This design also accommodates the attachment of additional accessories, offering versatility in the scope of medical examinations that can be performed with the device.

In FIG. 8B, a sectional view of the connecting part (10) as seen in FIG. 8A is detailed, showcasing the extension (17), protrusions, and recess-shaped structures which can be modularly attached to the main body of the connecting part (10). These components are designed with surfaces that may reflect light, thereby optimizing the distribution of illumination across the examination area. Their design allows for a customizable assembly to suit specific user needs or device configurations.

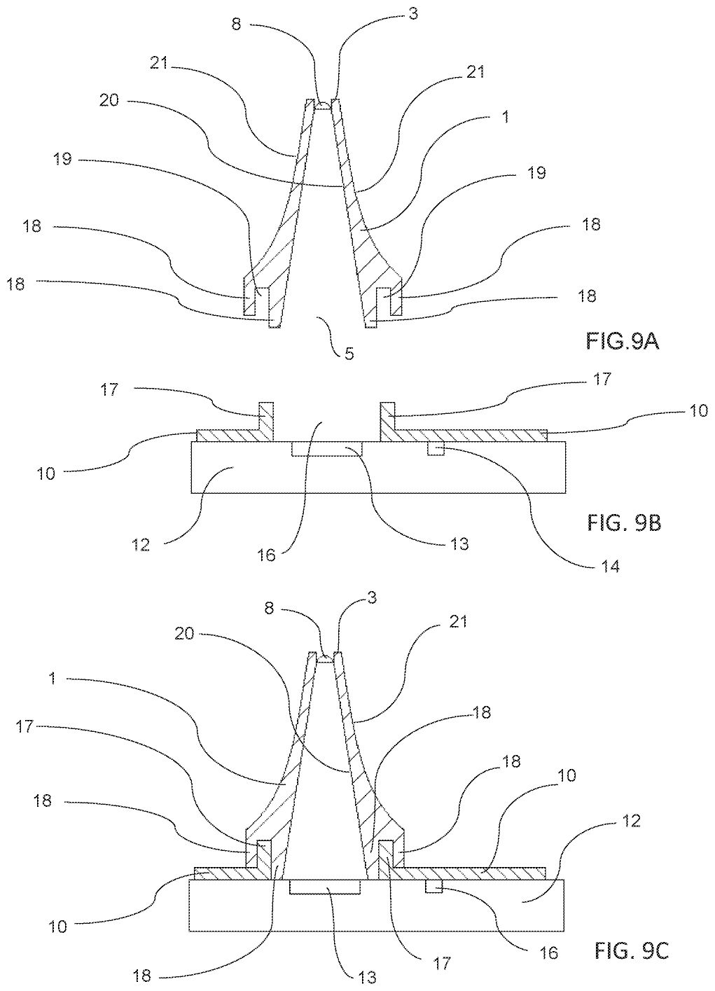

FIG. 9A depicts a side-sectional view of the speculum (1), focusing on the base part (2) equipped with light-modulating protrusions (18) and recesses (19). These features are engineered to enhance the connection between the speculum (1) and the connecting part (10), facilitating a seamless transmission and reflection of light within the device. The interface between the connector part (10) and the speculum (1) showcases a design that can be either symmetrical or asymmetrical, ensuring a snug fit and efficient light conduction. The speculum (1) itself is constructed to be light-conducting, with lenses (8) strategically placed to optimize visual clarity. Additionally, the surfaces of the speculum (1), both internal (20) and external (21), may be coated with materials that further enhance light reflection, improving the quality of the images captured.

FIG. 9B provides a side-sectional view illustrating the connection of the part (10) to the electronic device (12). The connecting part (10) can be directly affixed to the device (12) using various mechanisms that might include adhesive protrusions, indentations, or layers to ensure a secure yet reversible attachment. This allows the connecting part (10) to closely envelop the device (12), either making direct contact or establishing a connection with a slight separation, designed to comprehensively surround the device's light source (14). This strategic placement ensures optimal use of the electronic device's existing light capabilities, enhancing the effectiveness of the otoscope attachment.

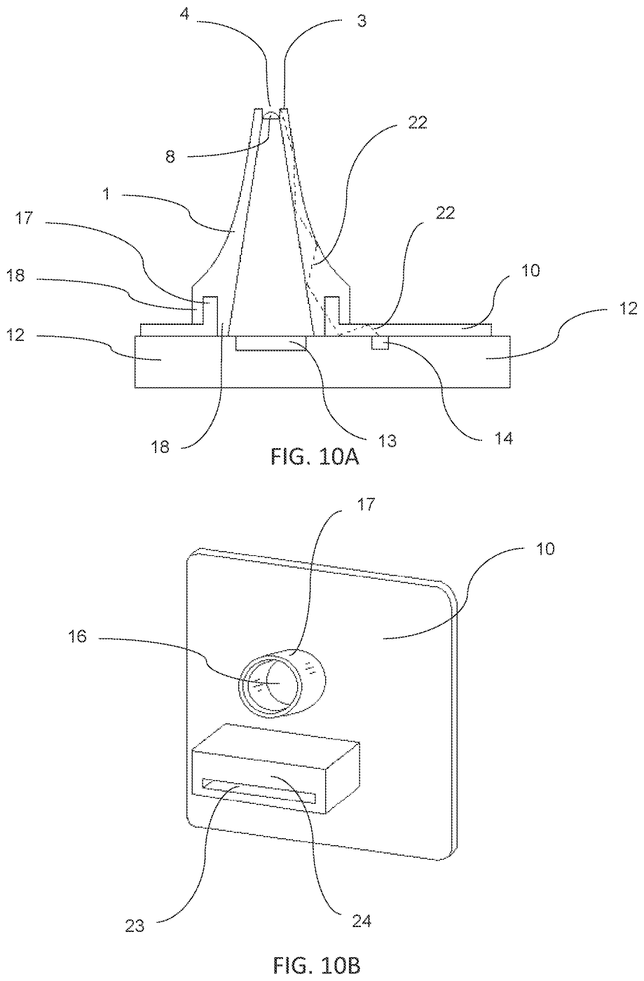

FIGS. 9C and 10A present cross-sectional views highlighting the assembly of the speculum (1) with the connecting part (10) mounted on the electronic device (12). This configuration ensures a precise alignment between the camera (13) of the device (12), the camera aperture (16) on the connecting part (10), and the speculum's opening (4). The design accommodates direct contact between the speculum (1), connecting part (10), and the electronic device (12), or alternatively, allows for a slight separation facilitated by intermediary layers. Regardless of direct contact or the presence of a gap, the critical alignment between the device's camera (13) and the speculum (1) remains consistent, ensuring optimal image capture. Moreover, the connecting part (10) plays a vital role in guiding light from the device's source (14) towards the front of the speculum (1).

In detail, FIG. 10A illustrates how the interconnected speculum (1) and connecting part (10) manage and refine the light sourced from the light source (14). Utilizing their inherent light-transmitting capabilities, these components guide the light towards the speculum's tip (3), as indicated by dashed lines (22), effectively illuminating the examination area. To enhance the efficiency of this light transmission, specific surfaces within the speculum (1) and on the connecting part (10) are treated with reflective materials. This reflective coating is meticulously applied to the inner surface (20), particularly between the lens (8) and the camera (13), to mitigate any potential glare effects from the light source (14), ensuring clarity and precision in the captured images. The external surfaces (21) of these components also feature a reflective treatment to minimize light dispersion, thereby concentrating illumination where it is most needed. The transparency of the tip of the speculum (3) and sections of the connecting part (10) further aids in the seamless transmission of light, culminating in a well-illuminated visual field for the user. This thoughtful integration of light-management features within the speculum (1) and connecting part (10) underscores the invention's commitment to providing clear, well-lit views of the examination area, enhancing diagnostic capabilities.

FIG. 10B offers a perspective view of the connecting part (10), highlighting its versatile design that accommodates the attachment of various medical accessories through an array of connection mechanisms including protrusions, clips, adhesive areas, and magnetic interfaces. This flexibility enables the connecting part (10) to support a wide range of diagnostic tools, enhancing the utility of the electronic device (12) it is paired with. For instance, dermatoscopes or ophthalmoscopes equipped with specialized lenses for directing light can be securely attached to the connecting part (10) for detailed skin or eye examinations, respectively. Additionally, attachments such as tongue depressors can be connected at specific points, like point (23), with the aid of a protrusion (24) designed to channel light towards the throat for clear visualization. Similarly, a vaginal speculum with the capability to transmit light can be attached to effectively illuminate and examine the cervix area, although specific visualization of this configuration is not depicted in the illustrations. The design also accommodates the attachment of otoscope specula (1) directly to the camera opening (16), ensuring a seamless integration with the electronic device's (12) imaging capabilities.

Furthermore, the connecting part (10) incorporates a dynamic platform capable of repositioning attached accessories either through rotational movement around a central point or via horizontal sliding. This adaptability allows for precise alignment of the accessories with the camera opening (16) and the light source (14), optimizing the functionality of each attached tool based on its specific requirements. The innovative design of the connecting part (10) not only secures these accessories in place but also facilitates the effective transmission of light from the light source (14) to the examination area. In essence, the connecting part (10) serves a multifaceted role, acting as a modular hub that not only secures but also enhances the functionality of various diagnostic accessories by providing essential lighting and imaging support, thereby expanding the capabilities of the standard electronic device (12) it complements.

The components constituting the otoscope, including the speculum (1) and connecting part (10), are designed for flexibility in assembly and manufacturing. These elements may be created as independent units to be assembled post-production or fabricated as a unified structure. The manufacturing processes employed can range from molding and plastic injection for the creation of polymer-based parts to the use of molding or CNC (Computer Numerical Control) cutting techniques for the crafting of metal and glass components. The lenses (8), integral for image clarity and focus, are made from materials such as glass, plastic, or silicone derivatives, offering various optical properties to suit specific diagnostic needs. These components can either be manufactured in one piece or produced separately and subsequently assembled using adhesives, ensuring structural coherence and operational integrity.

Incorporated within the otoscope is a sophisticated artificial intelligence (AI) framework designed to augment the device's functionality by optimizing several critical parameters such as stability, lighting, distance, and zoom and cropping settings. This AI integration includes a triad of model types aimed at enhancing the diagnostic capabilities of the device: (1) classifiers dedicated to assessing the quality of captured images, ensuring that only high-quality visuals are utilized for diagnosis; (2) deep learning algorithms tailored to assist users in achieving optimal positioning of the device, thereby maximizing the efficacy of examinations; and (3) advanced pixel-level image fusion models that work to amalgamate and refine image data, producing exceptionally clear and detailed final images. These AI-driven features are crafted to adapt to various user requirements and examination contexts, enhancing the versatility and diagnostic precision of the otoscope.

The features detailed—ranging from the modular design of the otoscope's physical components to its AI-enhanced operational capabilities—may be employed individually or in combination across different implementations of the device. This modular and integrated approach ensures that the otoscope can be customized to meet diverse diagnostic needs, embodying a balance between advanced technological integration and user-centric design.

The entirety or each component comprising the otoscope (1) can be produced from biodegradable, compostable, or recyclable materials. Biodegradable plastics encompass polylactic acid (PLA), polyhydroxyalkanoates (PHA), polybutylene adipate terephthalate (PBAT), polycaprolactone (PCL), starch-based plastics, and bioplastic PET (Bio-PET). These materials, which are not limited to those mentioned, include the following characteristics: These materials, derived from renewable resources or modified from traditional plastics, offer environmentally friendly alternatives that naturally break down over time, reducing pollution and waste accumulation. Compostable materials may originate from organic matter, cellulose, or paper, while recyclable materials may include paper, glass, or metal. The stethoscope can also be produced as a combination of various materials. Production methods may include one or more of the following steps: injection molding, cutting, assembling, welding.

The production can be carried out using the following methods: The continuous flow biodegradable or recyclable plastic production method comprises several interconnected steps. Initially, biodegradable polymer pellets or granules are fed into a mixing chamber where they are combined with additives and modifiers to achieve desired material properties. The mixture is then conveyed into an extrusion unit equipped with a twin-screw extruder. Within the extruder, the polymer blend is heated, melted, and homogenized under controlled temperature and pressure conditions. Following extrusion, the molten polymer is directed into a shaping die assembly designed to impart specific geometries to the plastic products. The die assembly may include interchangeable molds or nozzles to facilitate the production of various shapes and sizes. As the plastic material exits the die, it undergoes rapid cooling and solidification, preserving the desired form. To enhance the biodegradability of the final products, the manufacturing process can incorporate additives such as bio-based fillers, enzymes, or microbial agents that accelerate decomposition in natural environments. These additives are carefully integrated into the polymer blend during mixing to ensure uniform distribution and effectiveness. Additionally, the continuous flow nature of the production line enables high throughput and efficiency, minimizing downtime and waste. Automated monitoring and control systems oversee key process parameters, ensuring consistent product quality and performance. The disclosed continuous flow biodegradable plastic production method offers a sustainable approach to plastic manufacturing, utilizing renewable resources and environmentally friendly practices to produce biodegradable plastics suitable for a wide range of applications.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the otoscope device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An otoscope device attachment for use with image capture devices comprising illumination capabilities, the device attachment comprising:
a connecting component configured to detachably secure to a user device, the connecting component including a planar body having a first side with means for attaching to the user device and an opposing second side, at least one central aperture being formed in the connecting component planar body for exposing a camera of the user device;
a speculum having an elongated body with an outer surface and an inner surface defining a straight tunnel extending from a proximal end to a distal end of the speculum, the proximal end forming a base configured to detachably couple to the second side of the connecting component, and the distal end forming a tip; and
one or more lenses positioned along the length of the straight tunnel within the speculum;
wherein the connecting component and the speculum are arranged such that, when the user device is installed in the connecting component with the speculum coupled thereto, a camera of the user device exposed by the central aperture is aligned with the straight tunnel of the speculum, and light from a light source adjacent to the camera of the user device is configured to travel along the length of the speculum from the base to the tip through a combination of apertures, transparency, and internal light reflection within the connecting component and the speculum.

2. The otoscope device attachment of claim 1, wherein the means for attaching the connecting component to the user device comprises adhesive materials, suction cups, or clip-shaped structures.

3. The otoscope device attachment of claim 1, wherein the speculum is composed of an inner body and an outer body, the inner body forming the inner surface of the straight tunnel, and the outer body forming the outer surface, with a hollow space between them for facilitating internal light reflection.

4. The otoscope device attachment of claim 3, wherein at least one of the inner body and the outer body of the speculum is made from a material selected from the group consisting of plastic, metal, silicone, biodegradable materials, and glass, and is either transparent, translucent, or opaque.

5. The otoscope device attachment of claim 3, wherein the outer wall of the inner body and the inner wall of the outer body have reflective properties to enhance the transmission and reflection of light along the length of the speculum.

6. The otoscope device attachment of claim 1, wherein the lenses are selected from convex, concave, or a combination thereof, and are configured for transmitting images from the tip of the speculum to the camera of the user device in a clear manner.

7. The otoscope device attachment of claim 6, wherein the lenses are coated with various light filters to enhance the quality of the transmitted images.

8. The otoscope device attachment of claim 1, wherein the speculum has a base wider than the tip, taking on a tapered shape to facilitate easy insertion into an ear, nose, or other body orifice.

9. The otoscope device attachment of claim 1, wherein the connecting component is configured to be compatible with electronic devices of different sizes and shapes, ensuring the proper alignment of the camera with the central aperture regardless of the device model.

10. The otoscope device attachment of claim 1, wherein the connecting component further comprises shapes and apertures designed to match the camera's image area, ensuring that the camera captures images through these apertures while allowing light from the light source to pass through.

11. The otoscope device attachment of claim 1, further comprising a mechanism within the connecting component for selecting which camera on a multi-camera electronic device is to be used with the attachment.

12. The otoscope device attachment of claim 1, wherein the speculum comprises a soft material at its distal end, designed to safely and comfortably enter an orifice without causing harm.

13. The otoscope device attachment of claim 1, wherein the connecting component is equipped with multiple connection points including one or more of: protrusions, clips, adhesive surfaces, and magnetic surfaces for attaching a variety of medical examination accessories.

14. The otoscope device attachment of claim 13, further comprising a rotatable platform on the connecting component, capable of positioning different accessories in alignment with the camera opening and the light source.

15. The otoscope device attachment of claim 13, wherein the connection part includes a mechanism for linear movement, allowing accessories to move back and forth to adjust the opening or closing of the camera and light source.

16. The otoscope device attachment of claim 1, integrated with artificial intelligence models to automatically adjust stability, lighting, distance, and zoom for optimizing image capture, wherein the AI models include classifiers for assessing image quality, deep learning models for guiding patient positioning, and pixel-level image fusion models for enhancing image resolution and clarity.

17. A continuous flow biodegradable plastic production method comprising:
mixing biodegradable polymer materials with additives and modifiers in a mixing chamber;
extruding the polymer blend using a twin-screw extruder under controlled temperature and pressure conditions;
shaping the molten polymer into desired products using a shaping die assembly;
incorporating biodegradability-enhancing additives into the polymer blend during mixing to accelerate decomposition in natural environments;
automating monitoring and control systems to ensure consistent product quality and performance.
